(19) United States
(12) Patent Application Publication
(10) Pub. No.: US 2020/0205709 A1
(43) Pub. Date: Jul. 2, 2020

(54) MENTAL STATE INDICATOR

(71) Applicant: MEDIBIO LIMITED, South Yarra, Victoria (AU)

(72) Inventors: Yashar BEHZADI, South Yarra (AU); Nathan KOWAHL, South Yarra (AU); Matthew WESCOTT, South Yarra (AU); Nick HUGHES, South Yarra (AU); Sangyeop LEE, South Yarra (AU)

(73) Assignee: MEDIBIO LIMITED, South Yarra, Victoria (AU)

(21) Appl. No.: 16/621,644

(22) PCT Filed: Jun. 11, 2018
(86) PCT No.: PCT/AU2018/050578

§ 371 Date: Dec. 11, 2019

Related U.S. Application Data
(60) Provisional application No. 62/518,389, filed on Jun. 12, 2017.

(51) Int. Cl. A61B 5/16; G16H 50/20; G16H 50/70; G16H 50/50; A61B 5/024; A61B 5/00

(52) U.S. Cl. CPC A61B 5/16; G16H 50/20; G16H 50/70; A61B 5/0402; A61B 5/02405; A61B 5/4809; G16H 50/50

(57) ABSTRACT

A monitoring system for generating a mental state indicator for use in identifying a mental state of a biological subject, including one or more electronic processing devices that obtain subject data indicative of at least a heart rate measured for the biological subject during at least part of a sleep episode, analyze the subject data to determine at least one sleep segment; analyze the subject data to determine at least one metric for the at least one sleep segment, and apply the at least one metric to a computational model to determine a mental state indicator indicative of a mental state, the computational model embodying a relationship between different mental states and one or more metrics, and being obtained by applying machine learning to reference metrics derived from heart rates measured for one or more reference subjects during at least part of a reference sleep period.

### BACKGROUND OF THE INVENTION

The present invention relates to a method and apparatus for determining a mental state indicator for use in identifying a mental state of a subject.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

It is estimated that one in four citizens of developed nations will experience a mental health problem during their lifetime, with up to 10 percent of the population experiencing some type of depressive or anxiety-related disorder every year. The global economic cost of mental illness is measured in trillions of dollars annually.

Presently, there is no accepted and widely recognised objective test for many mental illnesses, such as depression. The diagnostic ‘gold standard’ in such cases remains clinical/expert assessment and opinion, based upon interviews with the patient along with close friends and family, and self-reporting (e.g. through the completion of questionnaires), for comparison against clinical symptoms catalogued in the Diagnostic and Statistical Manual of Mental Disorders (currently DSM-5).

However, due to the subjective nature of many aspects of this diagnostic process, agreement between clinicians can vary considerably, even for high-prevalence disorders such as depression and anxiety.

There is, accordingly, a need for quantitative, objective tests that can be employed by clinicians when identifying psychological disorders, and for monitoring the progress of patients undergoing treatment. Ideally, such tests should be simple, safe and unobtrusive, so that they can be undertaken without significant impact on the patient's lifestyle or day-to-day routine.

Provision of objective tests for mental health would enable numerous significant benefits to be realised. Better objective information could lead to earlier diagnosis, earlier intervention, and better outcomes for patients. Ongoing monitoring of patients could provide an objective indication of therapeutic effectiveness, enabling treatments to be varied and optimised based upon patient response. These improvements in treatment and outcomes would result in savings to the health system, and to the community in general.

It has been known for some time that there is a relationship between circadian heart rate patterns and psychological state. For example, U.S. Pat. No. 6,245,021 describes the use of recorded 24-hour heart rate patterns in the diagnosis of psychological disorders including depression, anxiety, panic disorder, obsessive compulsive disorder (OCD) and schizophrenia. However, the procedures disclosed in this patent still require expert (i.e. human) review of circadian heart rate patterns, by clinicians with the necessary training and experience to identify features that are commonly associated with the different disorders. Patients are required to maintain a daily diary, which enables the clinician to compare features in the heart rate patterns against activity (e.g. exercise) in which the patient may have engaged, so as to avoid misinterpreting these features. Clearly, a system that requires 24-hour monitoring, and the keeping of a daily diary, has a noticeable impact upon the patient's lifestyle and day-to-day routine, leading to a greater likelihood of non-compliance with the measurement and monitoring regime.

Accordingly, it would be desirable to develop new and objective methods and systems to assist in identifying individuals who may be suffering from, or at risk of, adverse mental health conditions such as depression, and which are able to provide one or more of the benefits discussed above.

US2017/0119297 describes a computer-implemented method of assessing a mental state of a subject, which includes receiving, as input, a heartbeat record of the subject. The heartbeat record comprises a sequence of heartbeat data samples obtained over a time span which includes a pre-sleep period, a sleep period having a sleep onset time and a sleep conclusion time, and a post-sleep period. At least the sleep onset time and the sleep conclusion time are identified within the heartbeat record. A knowledge base is then accessed, which comprises data obtained via expert evaluation of a training set of subjects and which embodies a computational model of a relationship between mental state and heart rate characteristics. Using information in the knowledge base, the computational model is applied to compute at least one metric associated with the mental state of the subject, and to generate an indication of mental state based upon the metric. The indication of mental state is provided as output.

### SUMMARY OF THE PRESENT INVENTION

In one broad form, an aspect of the present invention seeks to provide a monitoring system for generating a mental state indicator for use in identifying a mental state of a biological subject, the monitoring system including one or more electronic processing devices that: obtain subject data indicative of at least a heart rate measured for the biological subject during at least part of a sleep episode; analyze the subject data to determine at least one sleep segment selected from the group including: n minutes preceding sleep onset; n minutes following sleep onset; the sleep episode; a first half of the sleep episode; a second half of the sleep episode; n minutes prior to waking; analyze the subject data to determine at least one metric for the at least one sleep segment, the at least one metric being selected from the metric group including: a heart rate statistic metric selected from a heart rate statistic group including: a mean; a median; an average; a variance; a skew; a kurtosis; a percentile; a cumulative distribution function; a heart rate spectral power metric indicative of a spectral power in at least one frequency band selected from a frequency band group including: an ultra low frequency less than about 0.003 Hz; a very low frequency between about 0.003 Hz and about 0.04 Hz; a low frequency between about 0.04 Hz and about 0.15 Hz; a high-frequency between about 0.15 Hz and about 0.4 Hz; a heart rate variability metric selected from a heart rate variability group including: a multi-scale entropy; a standard deviation of average pulse intervals; and, square root of the mean of the squares of differences between adjacent pulse intervals; and, apply the at least one metric to at least one computational model to determine an mental state indicator indicative of a mental state, the at least one computational model embodying a relationship between different mental states and one or more metrics, the at least one computational model being obtained by applying machine learning to reference metrics derived from heart rates measured for one or more reference subjects during at least part of a reference sleep period.

In one embodiment the one or more processing devices determine at least one of: at least one metric for each of a plurality of sleep segments; a plurality of metrics for at least one sleep segment; and, a plurality of metrics for each of a plurality of sleep segments.

In one embodiment the subject data is indicative of a sleep state for the biological subject during at least part of each of a number of sleep episodes and wherein the at least one metric includes a sleep metric selected from a sleep metric group including: a total sleep duration; a number of sleep episodes; a mean sleep episode duration; and, a standard deviation of sleep episode durations.

In one embodiment the sleep state is derived from at least one of: heart rate data indicative of the heart rate measured for the biological subject during at least part of a sleep period; brain activity data indicative of brain activity measured for the biological subject during at least part of a sleep period; and, activity data indicative of physical activity measured for the biological subject during at least part of a sleep period.

In one embodiment the subject data includes brain activity data indicative of brain activity measured for the biological subject during at least part of a sleep period and wherein the one or more processing devices: analyze the brain activity data to determine at least one sleep stage selected from the sleep stage group including: REM sleep; non-REM sleep stage 1; non-REM sleep stage 2; non-REM sleep stage 3; and, awake; and, determine at least one brain activity metric selected from a brain activity metric group including: an absolute time in each sleep stage; a fractional time in each sleep stage; a sleep stage latency; a mean heart-rate in each sleep stage; a difference of mean heart-rates in each sleep stage.

In one embodiment the one or more processing devices determines a plurality of metrics selected from: a heart rate statistic metric group; a heart rate spectral power metric group; a heart rate variability metric group; a brain activity metric group; and, a sleep metric group.

In one embodiment the one or more processing devices determine at least one of: at least one of metric from each available group; and, at least two metrics from at least some available groups.

In one embodiment the one or more processing devices determines at least one of: at least two metrics; at least three metrics; at least four metrics; at least five metrics; at least six metrics; at least seven metrics; at least eight metrics; at least nine metrics; and, at least ten metrics.

In one embodiment the one or more processing devices: determine one or more subject attributes from the subject data; and, use the one or more subject attributes to apply the at least one computational model so that the at least one metric is assessed based on reference metrics derived for one or more reference subjects having similar attributes to the subject attributes.

In one embodiment the one or more processing devices select a plurality of metrics at least in part using the subject attributes.

In one embodiment the one or more processing devices select at least one computational model at least in part using the subject attributes.

In one embodiment the one or more subject attributes are selected from an attribute group including: one or more subject characteristics selected from a characteristic group including: a subject age; a subject height; a subject weight; a subject sex; and, a subject ethnicity; one or more possible mental states selected from a mental state group including: healthy; abnormal; depression; anxiety; panic disorder; obsessive compulsive disorder (OCD); and, schizophrenia; one or more body states selected from a body state group including: a healthy body state; an unhealthy body state; and, one or more disease states; one or more medical symptoms selected from a medical symptom group including: elevated temperature; coughing; sneezing; bloating; abnormal bowel movement; and, nausea; one or more perceived emotional states selected from an emotional state group including: happy; sad; anxious; angry; tired; and, shocked; dietary information; and, medication information.

In one embodiment the one or more processing devices determine the subject attributes at least one of: by querying a subject medical history; by receiving sensor data from a sensing device; and, in accordance with user input commands.

In one embodiment the one or more processing devices: compare at least one current metric determined for the subject during one or more current sleep episodes and at least one previous metric determined for the subject during one or more previous sleep episodes; and, using results of the comparison to track a change in mental state.

In one embodiment the one or more processing devices perform the step of comparing the at least one current metric and the at least one previous metric by: applying the at least one current metric to the at least one computational model to determine a current mental state indicator indicative of a current mental state; applying the at least one previous metric to the at least one computational model to determine a previous mental state indicator indicative of a previous mental state; and, analysing a difference between the current and previous mental state indicators to determine the change in mental state.

In one embodiment an intervention is performed between the previous and current sleep episodes and the one or more processing devices determine an indication of an effectiveness of the intervention based on the change in mental state.

In one embodiment the mental state indicator is indicative of at least one of: a likelihood of the subject having a particular mental state; and, a likelihood of a severity of a particular mental state.

In one embodiment the mental state is selected from the mental state group including: normal; abnormal; depression; anxiety; panic disorder; obsessive compulsive disorder (OCD); and, schizophrenia.

In one embodiment the system includes a monitoring device including: at least one sensor; and, a monitoring device processor that generates sensor data in accordance with signals from the at least one sensor, the sensor data being indicative of at least one of: a heart rate of the subject; brain activity of the subject; and, physical activity of the subject.

In one embodiment the monitoring device is a wearable device.

In one embodiment the monitoring system includes a client device that: receives sensor data from the monitoring device; generates captured subject data including: a subject identifier indicative of an identity of the subject; and, at least one of: heart rate data indicative of the measured heart rate; brain activity data indicative of measured brain activity; and, activity data indicative of measured physical activity; and, transfers captured subject data to the one or more processing devices, the one or more processing being responsive to the captured subject data to incorporate this into the subject data using the identifier.

In one embodiment the client device: displays one or more questions; and, generates captured data at least in part in response to user input commands provided in response to the one or more questions.

In one embodiment the one or more processing devices at least one of: display a representation of the mental state indicator; store the mental state indicator for subsequent retrieval; and, provide the mental state indicator to a client device for display.

In one embodiment the at least one computational model has a discriminatory performance based on at least one of: an area under a receiver operating characteristic curve; an accuracy; a sensitivity; and, a specificity.

In one embodiment the discriminatory performance is at least 70%.

In one broad form, an aspect of the present invention seeks to provide a method for generating an mental state indicator for use in identifying a mental state of a biological subject, the method including in one or more electronic processing devices: obtaining subject data indicative of at least a heart rate measured for the biological subject during at least part of a sleep episode; analyzing the subject data to determine at least one sleep segment selected from the group including: n minutes preceding sleep onset; n minutes following sleep onset; the sleep period; a first half of the sleep period; a second half of the sleep period; n minutes prior to waking; analyzing the subject data to determine at least one metric for the at least one sleep segment, the at least one metric being selected from the metric group including: a heart rate statistic metric selected from a heart rate statistic group including: a mean; a median; an average; a variance; a skew; a kurtosis; a percentile; a cumulative distribution function; a heart rate spectral power metric indicative of a spectral power in at least one frequency band selected from a frequency band group including: an ultra low frequency less than 0.003 Hz; a very low frequency between 0.003 Hz and 0.04 Hz; a low frequency between 0.04 Hz and 0.15 Hz; a high-frequency between about 0.15 Hz and about 0.4 Hz; a heart rate variability metric selected from a heart rate variability group including: a multi-scale entropy; a standard deviation of average pulse intervals; and, square root of the mean of the squares of differences between adjacent pulse intervals; and, applying the at least one metric to at least one computational model to determine an mental state indicator indicative of a mental state, the at least one computational model embodying a relationship between different mental states and one or more metrics, the at least one computational model being obtained by applying machine learning to reference metrics derived from heart rates measured for one or more reference subjects during at least part of a reference sleep period.

In one broad form, an aspect of the present invention seeks to provide a system for use in calculating at least one computational model, the at least one computational model being used for generating an mental state indicator for use in identifying a mental state of a biological subject, the system including one or more electronic processing devices that: for each of a plurality of reference subjects: obtain reference subject data indicative of: at least a heart rate measured the reference subject during at least part of a reference sleep episode; and, a diagnosed mental state of the reference subject; analyze the reference subject data to determine at least one sleep segment selected from the group including: n minutes preceding sleep onset; n minutes following sleep onset; the sleep episode; a first half of the sleep episode; a second half of the sleep episode; n minutes prior to waking; analyze the reference subject data to determine at least one reference metric for the at least one reference sleep segment, the at least one metric being selected from the metric group including: a heart rate statistic metric selected from a heart rate statistic group including: a mean; a median; an average; a variance; a skew; a kurtosis; a percentile; a cumulative distribution function; a heart rate spectral power metric indicative of a spectral power in at least one frequency band selected from a frequency band group including: an ultra low frequency less than about 0.003 Hz; a very low frequency between about 0.003 Hz and about 0.04 Hz; a low frequency between about 0.04 Hz and about 0.15 Hz; a high-frequency between about 0.15 Hz and about 0.4 Hz; a heart rate variability metric selected from a heart rate variability group including: a multi-scale entropy; a standard deviation of average pulse intervals; and, square root of the mean of the squares of differences between adjacent pulse intervals; and, use the at least one reference metric and diagnosed mental state for a number of reference subjects to train at least one computational model, the at least one computational model embodying a relationship between different mental states and the at least one reference metric.

In one embodiment the one or more processing devices test the at least one computational model to determine a discriminatory performance of the model.

In one embodiment the discriminatory performance is based on at least one of: an area under a receiver operating characteristic curve; an accuracy; a sensitivity; and, a specificity.

In one embodiment the discriminatory performance is at least 70%.

In one embodiment the one or more processing devices test the at least one computational model using a reference subject data from a subset of the plurality of reference subjects.

In one embodiment the one or more processing devices: select a plurality of reference metrics; train at least one computational model using the plurality of reference metrics; test the at least one computational model to determine a discriminatory performance of the model; and, if the discriminatory performance of the model falls below a threshold, at least one of: selectively retrain the at least one computational model using a different plurality of reference metrics; and, train a different computational model.

In one embodiment the one or more processing devices: select a plurality of combinations of reference metrics; train a plurality of computational models using each of the combinations; test each computational model to determine a discriminatory performance of the model; and, selecting the at least one computational model with the highest discriminatory performance for use in determining an mental state indicator indicative of a mental state.

In one embodiment the one or more processing devices: determine one or more reference subject attributes from the reference subject data; train the at least one computational model using the one or more reference subject attributes.

In one embodiment the one or more processing devices: perform clustering using the using the reference subject attributes to determine clusters of reference subject having similar reference subject attributes; and, train the at least one computational model at least in part using the reference subject clusters.

In one broad form, an aspect of the present invention seeks to provide a method for use in calculating at least one computational model, the at least one computational model being used for generating an mental state indicator for use in identifying a mental state of a biological subject, the method including, in one or more electronic processing devices: for each of a plurality of reference subjects: obtaining reference subject data indicative of: at least a heart rate measured the reference subject during at least part of a reference sleep episode; and, a diagnosed mental state of the reference subject; analyzing the reference subject data to determine at least one sleep segment selected from the group including: n minutes preceding sleep onset; n minutes following sleep onset; the sleep episode; a first half of the sleep episode; a second half of the sleep episode; n minutes prior to waking; analyzing the reference subject data to determine at least one reference metric for the at least one reference sleep segment, the at least one metric being selected from the metric group including: a heart rate statistic metric selected from a heart rate statistic group including: a mean; a median; an average; a variance; a skew; a kurtosis; a percentile; a cumulative distribution function; a heart rate spectral power metric indicative of a spectral power in at least one frequency band selected from a frequency band group including: an ultra low frequency less than about 0.003 Hz; a very low frequency between about 0.003 Hz and about 0.04 Hz; a low frequency between about 0.04 Hz and about 0.15 Hz; a high-frequency between about 0.15 Hz and about 0.4 Hz; a heart rate variability metric selected from a heart rate variability group including: a multi-scale entropy; a standard deviation of average pulse intervals; and, square root of the mean of the squares of differences between adjacent pulse intervals; and, using the at least one reference metric and diagnosed mental state for a number of reference subjects to train at least one computational model, the at least one computational model embodying a relationship between different mental states and the at least one reference metric.

It will be appreciated that the broad forms of the invention and their respective features can be used in conjunction, interchangeably and/or independently, and reference to separate broad forms is not intended to be limiting.

### BRIEF DESCRIPTION OF THE DRAWINGS

Various examples and embodiments of the present invention will now be described with reference to the accompanying drawings, in which:—

FIG. 1 is a flow chart of an example of a method for generating a mental state indicator for use in identifying a mental state of a biological subject;

FIG. 2 is a flow chart of an example of a process for training at least one computational model;

FIG. 3 is a schematic diagram of an example of an apparatus for generating a mental state indicator for use in identifying a mental state of a biological subject;

FIG. 4 is a schematic diagram of an example of a network architecture;

FIG. 5 is a schematic diagram of an example of a processing system;

FIG. 6 is a schematic diagram of an example of a client device;

FIG. 7 is a flow chart of an example of a process for collecting subject data; and,

FIGS. 8A and 8E are a flow chart of a specific example of a method of generating a mental state indicator for identifying of a mental state of a biological subject.

### DETAILED DESCRIPTION OF THE PREFERRED EMBODIMENTS

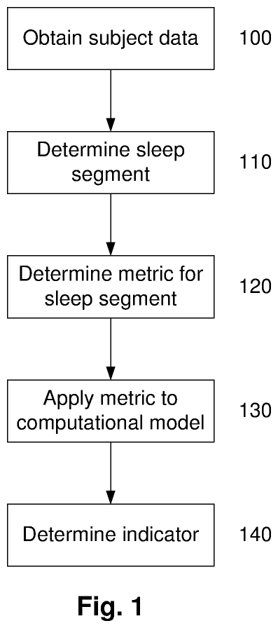

An example of the process for generating a mental state indicator for identifying a mental state of a biological subject will now be described with reference to FIG. 1.

For the purpose of this example, it is assumed that the method is performed at least in part using one or more electronic processing devices typically forming part of one or more processing systems, such as servers, personal computers or the like and which may optionally be connected to one or more processing systems, data sources or the like via a network architecture as will be described in more detail below.

For the purpose of explanation, the term “reference subject” is used to refer to one or more individuals in a sample population, with “reference subject data” being used to refer to data collected from the reference subjects. The term “subject” refers to any individual that is being assessed for the purpose of identifying a mental state, with “subject data” being used to refer to data collected from the subject. The reference subjects and subjects are animals, and more particularly humans, although this is not intended to be limiting and the techniques could be applied more broadly to other vertebrates and mammals.

In this example, at step 100 subject data is obtained which is at least partially indicative of a heart rate measured for a biological subject during at least part of a sleep episode.

The subject data could be obtained in any appropriate manner, including receiving data from a monitoring device, computer system or the like, retrieving the subject data from a data store such as a database, collecting at least some of the subject data using one or more sensors, or the like. The subject data typically includes heart rate data indicative of the heart rate, which may be in the form of simple pulse information but may also include Electrocardiography (ECG) data. The heart rate is typically measured for at least part of a sleep episode, but more typically over at least one entire sleep episode, which is a period of time during which the subject is asleep, between sleep onset and waking events. The subject data may also be indicative of a heart rate measured over multiple sleep episodes with the data optionally being collected continuously during the sleep episode or at periodic intervals through the sleep episode. The subject data may also include additional data, such as data regarding subject attributes or other physiological signals measured from the subject, such as measures of physical or mental activity, or the like, as will be described in more detail below.

At step 110 the subject data is analysed to determine at least one sleep segment. The sleep segment corresponds to a particular portion of a sleep episode and could include a number of minutes, such as 20 minutes, preceding or post sleep onset, an entire sleep episode, a first or second half of the sleep episode or a number of minutes prior to waking. The sleep segments can be determined in any appropriate manner depending upon the nature of the subject data available. For example, if the subject data includes brain wave activity data, such as electroencephalogram (EEG) data, sleep events such as sleep onset and waking can be detected based on brain wave patterns, with this then being used to determine sleep segments. Segments may also be determined from the sleep stage analysis, for example, determination of stage 1, 2, 3 and REM (rapid eye movement) sleep, using EEG data, as will be described in more detail below. Alternatively, if brain wave activity data is not available this may be determined from data relating to physical activity of the user, the heart rate of the user, or based on events such as manual notifications, or a combination of the above.

At step 120 the subject data is analysed to determine at least one metric for the at least one sleep segment. The metric(s) used will vary depending upon a range of factors, such as the at least one computational model to be used, subject attributes, the mental conditions being identified, or the like, as will be described in more detail below. Typically the metrics are selected from heart rate statistic metrics, heart rate spectral power metrics or heart rate variability metrics, with multiple metrics optionally being selected from across these groups.

Heart rate statistic metrics are based on the subject's heart rate and can include any one or more of a mean, a median, an average, a variance, a skew, a kurtosis, a percentile, a cumulative distribution function, or the like. Heart rate spectral power metrics are based on a spectral power of the subject's heart beats, and are typically assessed in frequency bands including, an ultra-low frequency band, such as less than about 0.003 Hz, a very low frequency band, such as between about 0.003 Hz and about 0.04 Hz, a low frequency band, such as between about 0.04 Hz and about 0.15 Hz or a high-frequency band, such as between about 0.15 Hz and about 0.4 Hz. The heart rate variability metric is based on the subject's heart rate and typically includes one or more of a multi-scale entropy, a standard deviation of average pulse intervals, a square root of the mean of the squares of differences between adjacent pulse intervals, or the like.

At step 130 the one or more metrics are applied to at least one computational model. For the remainder of the description, the term computational model will be understood to encompass one or more models and it will be understood that reference to a singular model is not intended to be limiting and could encompass using multiple models. The computational model typically embodies a relationship between different mental states and values of the one or more metrics, and can be obtained by applying machine learning to reference metrics derived from heart rates measured for one or more reference subjects during at least part of a reference sleep period. Thus, it will be appreciated that in practice reference subject data, equivalent to subject data, is collected for a plurality of reference subjects for which a variety of different mental states have been diagnosed. The collected reference subject data is used to calculate reference metrics, which are then used to train the computational model so that the computational model can discriminate between different mental states, based on metrics derived from at least the subject's heart rate. The nature of the computational model will vary depending on the implementation and examples will be described in more detail below.

The computational model is used to determine a mental state indicator indicative of a mental state at step 140. Typically the mental state indicator is indicative of a likelihood of the subject having a mental state, and may for example include a numerical value, for example indicating that the user has a 95% chance of suffering from depression. The mental state indicator could also be indicative of the likelihood of a plurality of mental states, for example indicating a 95% chance of depression and/or a 20% chance of suffering from anxiety. However, this is not necessarily essential, and it will be appreciated that any suitable form of mental state indicator could be used.

Accordingly, it will be appreciated that the above described method utilises a machine leaning technique in order to diagnose the likelihood of a subject having a one or more mental states utilising certain defined metrics relating to specific aspects of heart rate, heart rate spectral power or heart rate variability. The use of multiple metrics from the different groups can help improve the discriminatory performance of the computational model, in turn allowing mental states of the subject to be readily and accurately identified.

In one example, the particular metrics are used in a variety of combinations in order to provide computational models having a discriminatory performance, such as an accuracy, sensitivity, specificity or area under the receiver characteristic operating curve (AUROC) of greater than 70%. In this regard, a 70% performance metric is significant as this represents current best practice based on a psychiatric evaluation of the mental state of a subject by a trained psychiatrist, and far exceeds the ability of a general practitioner in assessing the mental state of a subject. Accordingly, in this case, the computational model exceeds the ability of general practitioners and can at least match the ability of trained psychiatrists. Providing a tool of this form therefore allows general practitioners, or other medical personnel, to substantially increase their effectiveness at identifying mental states of subjects, effectively bringing them up to at least being on par with trained psychiatrists.

A number of further features will now be described.

In one example, as previously mentioned, multiple metrics are used in order to increase the accuracy of the computational model, with this typically including at least one metric for each of a plurality of sleep segments, a plurality of metrics for at least one sleep segment, or a plurality of metrics for each of a plurality of sleep segments.

It will be further appreciated that metrics can be calculated across multiple sleep episodes, for example, to examine averages or variations of metrics between multiple sleep episodes, thereby reducing the likelihood of an unusual sleep episode leading to a misdiagnosis.

In addition to examining heart rate metrics, the system can examine other metrics to further enhance the discriminatory performance of the computational model, such as sleep and brain wave metrics.

In the case of sleep metrics, these are typically based on sleep states of the user over multiple sleep episodes, such as whether the subject is waking or sleeping, and can be used to derive metrics such as a total sleep duration, a total number of sleep episodes, a mean sleep episode duration, a standard deviation of sleep episode durations, or the like. The sleep states are typically determined using sleep data forming part of the subject data. The sleep data can be of any appropriate form and can be derived from a wide variety of data including heart rate data indicative of the heart rate, brain activity data indicative of brain activity, or activity data indicative of physical activity. It will also be appreciated that sleep states could be indicated via any other appropriate mechanism, such as based on information collected by a medical practitioner during a sleep study or the like.

When the subject data includes brain activity data, such as EEG data indicative of brain activity measured for the biological subject during part of a sleep period, this can be used to identify specific sleep stages, which are in turn used to determine brain activity metrics. For example, the brain activity data can be used to identify periods of REM sleep, non-REM sleep stage 1, non-REM sleep stage 2, non-REM sleep stage 3, and when the subject is awake. This information can then be used to determine at least one brain activity metric, such as an absolute time in each sleep stage, a fractional time in each sleep stage, a sleep stage latency, a mean heart rate in each sleep stage and a different in mean heart rates in each sleep stage. Thus, it will be appreciated that the above process can examine not only particular segments of a sleep episode, such as a period of time prior to sleeping, after sleeping or prior to waking, but also examines metrics associated with different sleep stages such as REM or non-REM sleep.

Typically, to maximise the effectiveness of the discriminatory performance of the computational model, the processing devices determine a plurality of metrics selected from across the groups including heart rate metric group, heart rate spectral power metric group, heart rate variability metric group, brain activity metric group and sleep metric group. The particular combination used will vary depending on a range of factors, including the availability to capture sleep and brain wave activity data, and the ability of the metrics to discriminate between different mental states. In one particular example, at least one metric is selected from each of the available groups and optionally at least two metrics are selected for at least some of the available groups. In general the number of metrics used include at least two, at least three, at least four, at least five, at least six, at least seven, at least eight, at least nine and in some cases at least ten.

The analysis is also typically performed to take into account subject attributes, such as subject characteristics, possible mental states suffered by the subject, or one or more subject body states. In this example, the one or more processing devices can use the one or more subject attributes to apply the computational model so that the at least one metric is assessed based on reference metrics derived for one or more reference subjects having similar attributes to the subject attributes. This can be achieved in a variety of ways, depending on the preferred implementation, and can include selecting metrics and/or one of a number of different computational models at least in part depending on the subject attributes. Irrespective of how this is achieved, it will be appreciated that taking into account subject attributes can further improve the discriminatory performance by taking into account that subjects with different attributes may react different to the same mental state. For example, a male 35 year old with depression may react differently to a female 60 year old with the same condition. Similarly, the assessment of depression could be quite different to the assessment of schizophrenia so different computational models and/or combinations of metrics could be selected depending on a mental state that is being identified.

The subject attributes could include subject characteristics such as a subject age, height, weight, sex or ethnicity, possible mental states from which the subject may suffer, including a normal or abnormal mental state, depression, anxiety, panic disorder, obsessive compulsive disorder (OCD), schizophrenia, or the like, body states, such as a healthy or unhealthy body states or one or more disease states, such as whether the subject is obese. The subject attributes could include one or more medical symptoms, such as an elevated temperature, coughing, sneezing, bloating, abnormal bowel movement, nausea, or the like, or one or more perceived emotional states, such as happy, sad, anxious, angry, tired, shocked, or the like. Finally, the subject attributes could include dietary information, such as details of any food or drink consumed, or medication information, including details of any medications taken.

The subject attributes could be determined in any one of a number of ways and could involve having the subject supply information a first time monitoring is performed, for example when registering to undergo the monitoring process, and/or periodically, for example each time monitoring is performed, at set time intervals, such as once a week or month, or the like. In this regard, a user interface could be displayed to the user by the processing device, or another computer system or client device, prompting the user to enter relevant information. This could be performed before or after each sleep session, with different information being requested each time, to avoid over burdening the subject. It will be appreciated that regular updates of subject attributes are typically used for more variable attributes, such as details of medical symptoms or the like, whilst attributes that are more static may be determined on a one off basis. It will also be appreciated that the processing devices could determine at least some of the subject attributes based on a subject medical history, for example by retrieving or querying a patient medical record.

The system can also be utilised to perform longitudinal monitoring in which changes in a subject's mental state are tracked. This can be performed for the purpose of monitoring of progression of a mental condition, as well as the efficacy of a treatment regime or program. In this example, a comparison is performed between at least one current metric determined for the subject during one or more current sleep episodes and at least one previous metric determined for the subject during one or more previous sleep episodes, with results of the comparison being used to track a change in a mental state. The comparison can be achieved by directly comparing particular metrics, but it will be appreciated that this tends to provide little in the way of guidance regarding the progression of the condition. Accordingly it is more typical for the current metric to be applied to the computational model to determine a current mental state indicator indicative of a current mental state, and to apply the previous metric to the computational model to determine a previous mental state indicator indicative of a previous mental state, and then analyse a difference between the current and previous mental state indicators to determine the change in mental state.

It will be appreciated that in practice, the system will maintain a record of mental state indicators, with each mental state indicator being generated over one or more sleep episodes, so that a sequence of mental state indicators can be used to demonstrate changes in the medical condition over time. In one example, such trending can be plotted allowing a medical practitioner to readily observe changes in mental state. For example this may demonstrate an increasing likelihood that the subject has a mental state, such as depression with the medical practitioner then being able to perform intervention, such as prescribing medication, with further changes such as a reduction in likelihood of depression being used to monitor effectiveness of treatment.

In a preferred example, the mental states are plotted contemporaneously with additional information, such as details of the timing of an intervention, including administering, or changing medication, allowing the response to the intervention to be easily tracked.

The mental state indicator can be indicative of a likelihood of a subject having a particular mental state or a likelihood of a severity of a particular mental state depending upon the preferred implementation and the nature of the mathematical model. The mental state indicator could be used in respect of a variety of mental states, such as normal and abnormal mental states, as well as specific conditions such as depression, anxiety, panic disorder, obsessive compulsive disorder and schizophrenia, or the like.

The nature of the monitoring system and in particular the hardware used to capture the subject data, and in particular the heart rate data and optionally any brain wave or activity data will vary depending upon the preferred implementation.

In one example, the monitoring system includes a monitoring device having at least one sensor and a monitoring device processor that generates sensor data in accordance with signals from the sensor. The sensor data is typically indicative of one or more of a heart rate of the subject, brain activity of the subject and physical activity of the subject, depending on the nature of the sensor employed.

In one example, the monitoring device is in the form of a wearable monitoring device which could include a wrist or chest mounted heart rate monitor, including a suitable heart rate detection mechanism. Examples include the use of an optical based system for detection of wrist pulse, or a movement sensor for detection of a chest pulse. Physical activity can be determined through the use of accelerometers or gyroscopes and may be incorporated into a wearable device. In one particular example, the monitoring device could include a wrist mounted smart watch or similar, with an optional chest strap for improved heart rate sensing.

Additionally and/or alternatively electrode based detection can be used to acquire ECG signals, which can in turn provide greater information regarding the heart rate and heart beat power. Similarly brain activity can be measured using an EEG sensing system, which may include electrodes attached to the scalp of the user, as part of a headset or the like.

It will be appreciated that the form factor of the monitoring device and the particular sensing provided can vary depending on the circumstances in which the monitoring device is to be used. For example, when used in a home environment, the monitoring device is typically a wearable device, with more limited sensing capabilities, often limited to optical and/or movement sensing, whereas if the device is adapted to be used in a clinical environment, such as during a sleep study, electrode based systems can be used for capturing ECG and EEG signals, for greater accuracy.

In one example, the monitoring device is adapted to upload sensor data directly to the one or more processing devices, which could be situated remotely in a cloud based environment, or locally, for example on a computer system. In another example, the monitoring system includes a client device, such as a smartphone, tablet or computer system, that receives sensor data from the monitoring device uses the sensor data to generate captured subject data. The captured subject data typically includes a subject identifier indicative of an identity of the subject, which could be a device identifier of either the client device or monitoring device, which is associated with the subject, or could be a user name or real name of the subject, or a unique identifier associated with the subject. The captured subject data further includes one or more of heart rate data indicative of the measured heart rate, brain activity data indicative of the measured brain activity or activity data indicative of measured physical data.

The captured subject data is transferred to the one or more processing devices, allowing these to incorporate the captured subject data into subject data using the identifier. Thus, the processing device can identify the stored subject data associated with the respective subject using the identifier, before updating the stored subject data with the captured subject data.

The client device can also be adapted to perform at least preliminary processing of the sensor data or this may alternatively be performed by the monitoring device itself. Such preliminary processing can include filtering of signals, derivation of parameters from the signals, such as to determine an inter-beat period, heartbeat frequency, power spectrums or the like. Although alternatively raw sensor data could be uploaded directly to the one or more processing devices as required.

Thus, in the above examples, the client device acts to acquire sensor data from the monitoring device, perform optional processing, and add an identifier, transferring this as captured subject data to the one or more processing devices, which are typically in the form of remote servers, allowing the subject data to be consolidated and processed remotely. In this example, the client device, which is typically a smart phone or tablet of the subject, effectively acts to forward the captured data to the processing devices for analysis as required.

The above described arrangement provides a number of benefits. For example, this ensures subject data is stored centrally, allowing this to be used in training computational models. This in effect allows data from multiple subjects to be mined so that more accurate models can be constructed thereby improving the discriminatory power of the system. Additionally, the client device can be used to leverage existing hardware functionality in order to reduce the hardware requirements of the monitoring device.

In a further example, the client device can be used to collect additional information, such as subject attributes. In this example, the client device can display one or more questions, generating the captured subject data at least in part in response to user input commands provided in response to the one or more questions. This allows the user to be presented with questions, which can in turn assist in assessment of the subject mental state, including capturing information relating to symptoms, as well as other information, such as questions regarding subject attributes, dietary habits, medication consumed, or the like.

In one example, the one or more processing devices display a representation of the mental state indicator, store the mental state indicator for subsequent retrieval or provide the mental state indicator to a client device for display. Thus, it will be appreciated that the mental state indicator can be used in a variety of manners, depending on the preferred implementation.

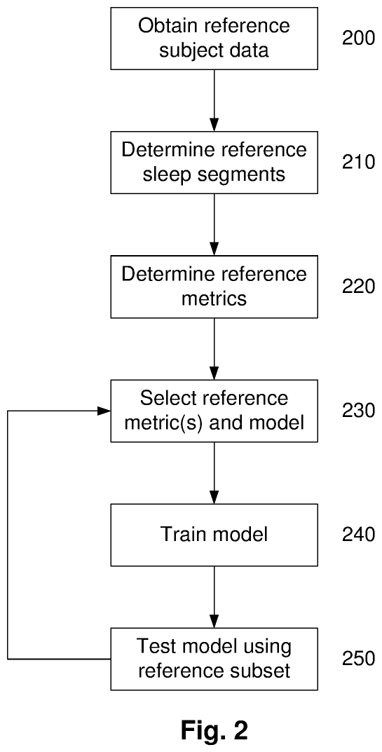

The above described approaches use a computational model in order to determine a mental state indicator, and an example of a process for generating such a model will now be described with reference to FIG. 2.

In this example, reference subject data is obtained at step 200, which is at least partially indicative of a heart rate measured for a reference subject during at least part of a reference sleep episode. At step 210 the reference subject data is analysed to determine at least one sleep segment corresponding to a particular portion of the reference sleep episode. At step 220 the reference subject data is analysed to determine at least one reference metric for the at least one reference sleep segment.

Steps 200 to 220 are largely analogous to steps 100 to 120 described with respect to obtaining and analysing subject data of a subject, and it will therefore be appreciated that these can be performed in a largely similar manner, and hence will not be described in further detail.

In contrast to subject data however, as the reference subject data is used in training a computational model, the reference subject data is also indicative of an identified mental state of the reference subject, which is typically obtained using a diagnosis by a medical practitioner, for example by receiving an indication of the diagnosis, retrieving this from a medical record, or the like.

Additionally, when using the reference subject data to train the computational model, it will be typically to determine reference metrics for all available metrics, rather than just selected ones of the metrics, allowing this to be used in order to ascertain which of the metrics are most useful in discriminating between different mental states. Nevertheless, the reference metrics used are as outlined above, and typically include heart rate statistic metrics, heart rate spectral power metrics, heart rate variability metrics, brain wave activity metrics or sleep metrics, as appropriate.

At step 230 a combination of the reference metrics and a generic computational model are selected, with the reference metrics and identified mental state for a plurality of reference subjects being used to train the model at step 240. The nature of the model and the training performed can be of any appropriate form and could include any one or more of decision tree learning, random forest, logistic regression, association rule learning, artificial neural networks, deep learning, inductive logic programming, support vector machines, clustering, Bayesian networks, reinforcement learning, representation learning, similarity and metric learning, genetic algorithms, rule-based machine learning, learning classifier systems, or the like. As such schemes are known, these will not be described in any further detail.

Accordingly, the above described process provides a mechanism to develop a computational model that can be used in generating a mental state indicator using the process described above with respect to FIG. 1.

In addition to simply generating the model, the process typically includes testing the model at step 250 to assess the discriminatory performance of the trained model. Such testing is typically performed using a subset of the reference subject data, and in particular, different reference subject data to that used to train the model, to avoid model bias. The testing is used to ensure the computational model provides sufficient discriminatory performance. In this regard, the discriminatory performance is typically based on an accuracy, sensitivity, specificity and AUROC, with a discriminatory performance of at least 70% being required in order for the model to be used.

It will be appreciated that if the model meets the discriminatory performance, it can then be used in determining a mental state indicator using the process outlined above with respect to FIG. 1. Otherwise, the process returns to step 230 allowing different metrics and/or models to be selected, with training and testing then being repeated as required.

Thus, in one example, the one or more processing devices select a plurality of reference metrics, typically selected as a subset of each of the metrics listed above, train a computational model using the plurality of reference metrics, test the computational model to determine a discriminatory performance of the model and if the discriminatory performance of the model falls below a threshold then selectively retrain the computational model using a different plurality of reference metrics and/or train a different computational model. Accordingly, it will be appreciated that the above described process can be performed iteratively utilising different metrics and/or different computational models until a required degree of discriminatory power is obtained.

As an alternative, the one or more processing devices can select a plurality of combinations of reference metrics, train a plurality of computational models using each of the combinations, test each computational model to determine a discriminatory performance of the model and select the computational model with the highest discriminatory performance for use in determining an mental state indicator indicative of a mental state.

In addition to use the metrics to train the models, the training can also be performed taking into account reference subject attributes, so that models are specific to respective reference subject attributes or can take the subject attributes into account when determining the mental state. In one example, this process involves having the one or more processing devices perform clustering using the using the reference subject attributes to determine clusters of reference subjects having similar reference subject attributes, for example using a clustering technique such as k-means clustering, and then training the computational model at least in part using the reference subject clusters. For example clusters of reference individuals suffering from depression could be identified, with this being used to train a computational model to identify depression. It will be appreciated however that any suitable technique could be used.

Accordingly, the above described techniques provide a mechanism for training one or more computational models to discriminate between different mental states using a variety of different metrics, and then using the model(s) to generates mental state indicators indicative of the likelihood of a subject having a particular mental state, thereby assisting in the diagnosis of mental states.

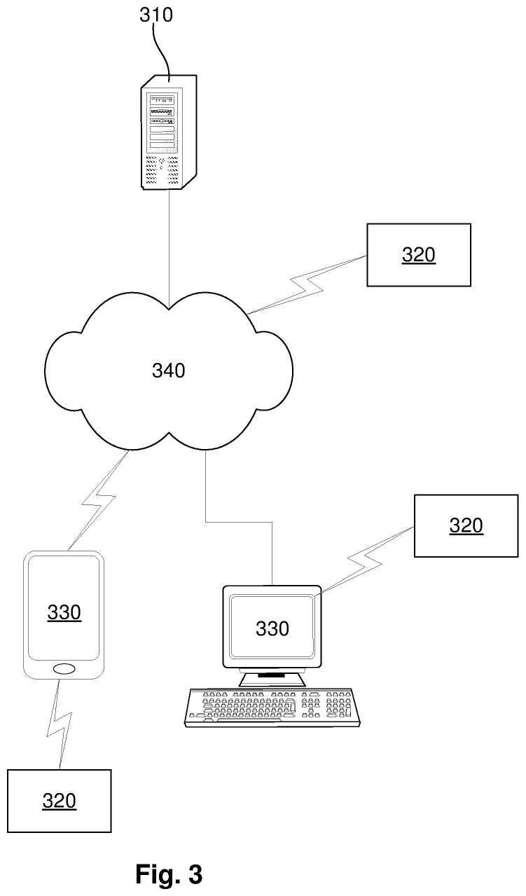

An example of a monitoring system will now be described in more detail with reference to FIG. 3.

In this example, one or more processing systems 310 are provided coupled to one or more client devices 330, via one or more communications networks 340, such as the Internet, and/or a number of local area networks (LANs). A number of monitoring devices 320 are provided, with these optionally being connected directly to the processing systems 310 via the communications networks 340, or more typically, with these being coupled to the client devices 330.

Any number of processing systems 310, monitoring devices 320 and client devices 330 could be provided, and the current representation is for the purpose of illustration only. The configuration of the networks 340 is also for the purpose of example only, and in practice the processing systems 310, monitoring devices 320 and client devices 330 can communicate via any appropriate mechanism, such as via wired or wireless connections, including, but not limited to mobile networks, private networks, such as an 802.11 networks, the Internet, LANs, WANs, or the like, as well as via direct or point-to-point connections, such as Bluetooth, or the like.

In this example, the processing systems 310 are adapted to receive and analyse subject data received from the monitoring devices 320 and/or client devices 330, allowing computational models to be generated and used to determine mental state indicators, which can then be displayed via the client devices 330. Whilst the processing systems 310 are shown as single entities, it will be appreciated they could include a number of processing systems distributed over a number of geographically separate locations, for example as part of a cloud based environment. Thus, the above described arrangements are not essential and other suitable configurations could be used.

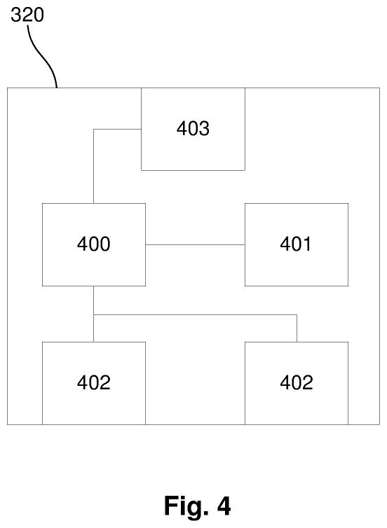

An example monitoring device 320 is shown in FIG. 4. In this example the monitoring device 320 includes a monitoring device processor 400 coupled to a monitoring device memory 401 and one or more sensors 402. The monitoring device processor is adapted to receive signals from the sensor(s) 402 and perform tasks in accordance with instructions stored in the memory 401, including optionally processing sensor data, for example by filtering and/or digitising sensor data, before transmitting the sensor data via transceiver 403 to either the processing system 310 or the client device 330.

Accordingly, it will be appreciated that the processing system monitoring device 320 may be formed from any suitable monitoring device, and could include a generic programmed monitoring device processor 400 such as a microprocessor, microchip processor, logic gate configuration, firmware optionally associated with implementing logic such as an FPGA (Field Programmable Gate Array), or any other electronic device, system or arrangement, and/or could include custom hardware, including specific arrangements for processing signals and generating sensor data as required.

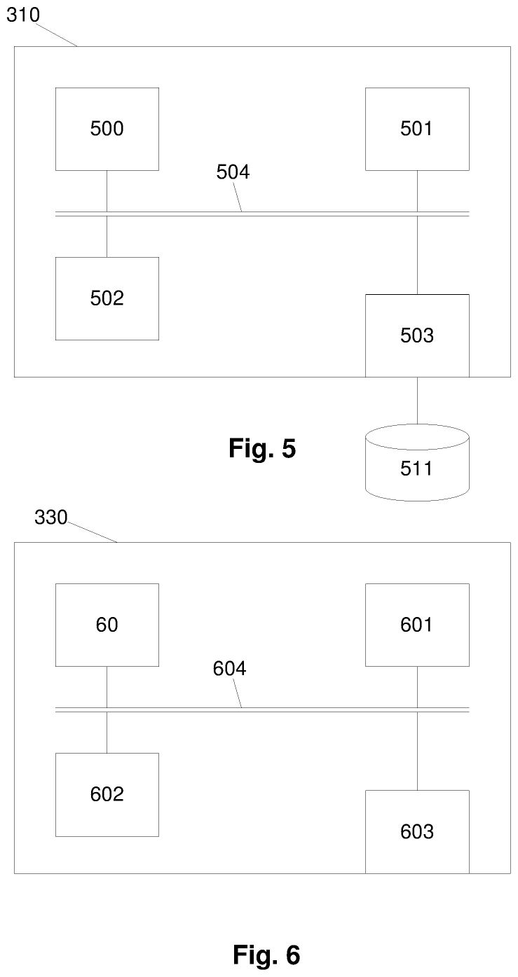

An example of a suitable processing system 310 is shown in FIG. 5. In this example, the processing system 310 includes at least one microprocessor 500, a memory 501, an optional input/output device 502, such as a keyboard and/or display, and an external interface 503, interconnected via a bus 504 as shown. In this example the external interface 503 can be utilised for connecting the processing system 310 to peripheral devices, such as the communications networks 340, databases 511, other storage devices, or the like. Although a single external interface 503 is shown, this is for the purpose of example only, and in practice multiple interfaces using various methods (eg. Ethernet, serial, USB, wireless or the like) may be provided.

In use, the microprocessor 500 executes instructions in the form of applications software stored in the memory 501 to allow the required processes to be performed. The applications software may include one or more software modules, and may be executed in a suitable execution environment, such as an operating system environment, or the like.

Accordingly, it will be appreciated that the processing system 510 may be formed from any suitable processing system, such as a suitably programmed PC, web server, network server, or the like. In one particular example, the processing system 510 is a standard processing system such as an Intel Architecture based processing system, which executes software applications stored on non-volatile (e.g., hard disk) storage, although this is not essential. However, it will also be understood that the processing system could be any electronic processing device such as a microprocessor, microchip processor, logic gate configuration, firmware optionally associated with implementing logic such as an FPGA (Field Programmable Gate Array), or any other electronic device, system or arrangement.

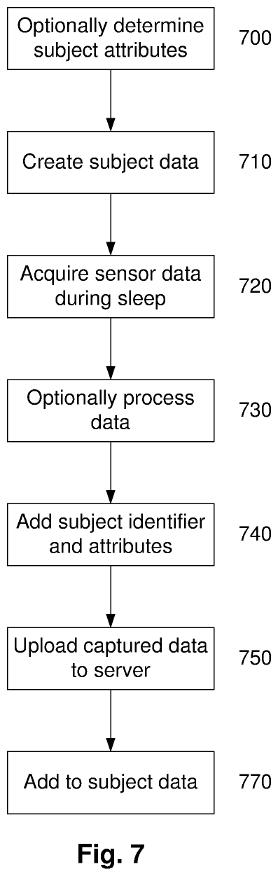

As shown in FIG. 6, in one example, the client device 330 includes at least one microprocessor 600, a memory 601, an input/output device 602, such as a keyboard and/or display, an external interface 603, interconnected via a bus 604 as shown. In this example the external interface 603 can be utilised for connecting the client device 330 to peripheral devices, such as the communications networks 340, databases, other storage devices, or the like. Although a single external interface 603 is shown, this is for the purpose of example only, and in practice multiple interfaces using various methods (eg. Ethernet, serial, USB, wireless or the like) may be provided. The card reader 604 can be of any suitable form and could include a magnetic card reader, or contactless reader for reading smartcards, or the like.

In use, the microprocessor 600 executes instructions in the form of applications software stored in the memory 601, and to allow communication with one of the processing systems 310 and/or monitoring devices 320.

Accordingly, it will be appreciated that the client device 330 be formed from any suitably programmed processing system and could include suitably programmed PCs, Internet terminal, lap-top, or hand-held PC, a tablet, a smart phone, or the like. However, it will also be understood that the client device 330 can be any electronic processing device such as a microprocessor, microchip processor, logic gate configuration, firmware optionally associated with implementing logic such as an FPGA (Field Programmable Gate Array), or any other electronic device, system or arrangement.

Examples of the processes for generating mental state indicators will now be described in further detail. For the purpose of these examples it is assumed that one or more respective processing systems 310 are servers adapted to receive and analyse subject data, and generate and provide access to mental state indicators. The servers 310 typically execute processing device software, allowing relevant actions to be performed, with actions performed by the server 310 being performed by the processor 500 in accordance with instructions stored as applications software in the memory 501 and/or input commands received from a user via the I/O device 502. It will also be assumed that actions performed by the client devices 330, are performed by the processor 600 in accordance with instructions stored as applications software in the memory 601 and/or input commands received from a user via the I/O device 602, whilst actions performed by the monitoring devices 320, are performed by the processor 400 in accordance with instructions stored as applications software in the memory 401 and/or input commands received from a user.

However, it will be appreciated that the above described configuration assumed for the purpose of the following examples is not essential, and numerous other configurations may be used. It will also be appreciated that the partitioning of functionality between the different processing systems may vary, depending on the particular implementation.

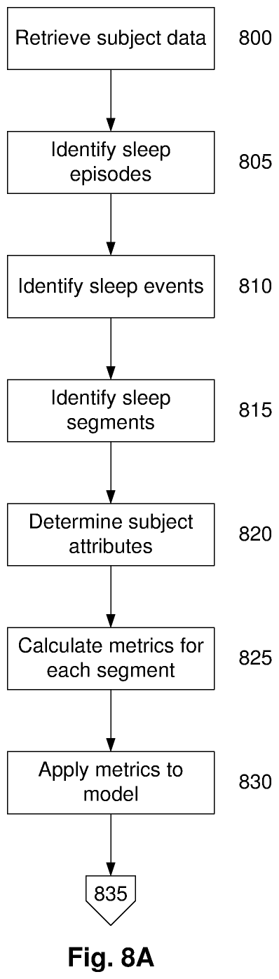

An example of the process for capturing subject data, or reference subject data, will now be described in more detail with reference to FIG. 7.

In this example, at step 700 information regarding one or more subject attributes is determined. This is typically performed as a one-off process when a subject undergoes an initial assessment, but could also be repeated periodically, such as each time a measurement is performed, depending on the nature of the subject attributes. The subject attributes could be retrieved by the server 310 from a medical record, for example, by providing the server 310 with details of the subject, allowing this to be used to retrieve the attributes, or could be provided by having a user, such as the subject or a supervising medical practitioner, enter the subject attributes via a suitable user interface presented on the client device 330. It will be appreciated that this could be achieved by presenting an application on the client device 330 and/or through a website hosted by the server 310.

At step 710, the server 310 generates subject data in the form of a particular record associated with the respective subject. As part of this process, a subject identifier is typically created and associated with the subject data. This could be a username, but more typically is a unique alphanumeric code, which can be used to anonymise the subject data as needed. The subject data may be stored in an encrypted database, with access permissions being defined for specific users, such as authorised medical practitioners, or the like, to thereby prevent unauthorised access to the subject data.

At step 720 sensor data is acquired by the monitoring device 320 whilst the subject is asleep. This would typically involve having the subject, or a medical practitioner, attach the monitoring device 320 to the subject in an appropriate manner, depending for example on the nature of the sensors 402. This could include simply having the subject wear the monitoring device 320, or could include attachment of electrodes to the subject. The sensor data is acquired, typically by filtering and digitising signals received from the sensors 402, before being uploaded to the client device 330.

At step 730, the client device can optionally process the received sensor data, for example to perform filtering and/or derive relevant parameters, such as a heart beat, or the like. Such parameterisation can reduce the volume of data that needs to be transferred to the server 310, although it will be appreciated that this is not essential, and alternatively the raw data could be transferred, depending on the preferred implementation.

At step 740, the client device 330 adds a subject identifier indicative of an identity of the subject to the sensor data together with any additional attribute information, to generate captured subject data.

This process may require that the client device 330 authenticate the subject, for example by having the subject provide biometric information, such as a fingerprint, or respond to an authentication challenge, for example by providing as password, enter a PIN (personal identification number) or the like. The subject identifier can then be retrieved from local memory on the client device 330, retrieved from the monitoring device 320, or could be entered manually by a user. Additionally, the process may require the client device 730 display one or more questions to the subject, allowing the subject to respond and provide information regarding attributes, such as current symptoms, details of any food, beverage or medications consumed or the like. The client device may also interface with other sensing devices, such as weight scales, allowing other subject attributes to be captured as required.

At step 750, the captured subject data is uploaded to the server 310, allowing this to be added to the subject data at step 760.

It will be appreciated that this process could be performed for subject undergoing assessment as well as reference subjects when the reference data is being collected to perform training of the computational model.

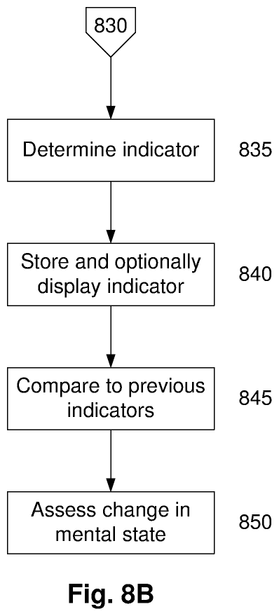

An example of the process of for analysing subject data for an individual will now be described in more detail with reference to FIGS. 8A and 8B.

In this example, at step 800 the server 310 obtains subject data, either retrieving this from a stored record or receiving this from a monitoring device 320, optionally via a client device 330, depending upon the preferred implementation.

At step 805 sleep episodes within the subject data are identified with individual sleep events within each sleep episode being identified at step 810. The identification of sleep episodes and sleep events is typically performed on the basis of EEG data where available and if not available activity data and/or heart rate data again depending on availability. In this regard EEG data typically provides great discriminatory power to identify sleep episodes and specific sleep events, such as onset sleep and waking, as well as different sleep stages, such as REM or non-REM sleep. In the event that EEG data is not available, which is typically only collected in a sleep clinic, the heart rate and an indication of physical activity can be used as a substitute.

Having identified sleep events, and particularly sleep onset and waking events, these is used to identify specific sleep segments including 20 minutes preceding sleep onset, 20 minutes following sleep onset, the entire sleep episode, a first half of the sleep episode, a second half of the sleep episode and 20 minutes prior to waking.

At step 820, the server 310 determines subject attributes for the subject from the subject data. The subject attributes can be used for selecting a computational model to be used and/or may be combined with the metrics in order to allow the computational model to be applied. In this regard, the metrics for the subject are typically analysed based on reference metrics for reference subjects having similar attributes to the subject. This could be achieved by using different computational models for different combinations of attributes, or by using the attributes as inputs to the computational model.

Accordingly, the server 310 then calculates relevant metrics for each sleep segment at step 825, with the relevant metrics being determined based on the requirements of the computational model.

At step 830 metrics are applied to the computational model, for example by using the relevant metrics, optionally together with one or more subject attributes, to form a feature vector, which is then applied to the computation model. The model analyses the feature vector, and calculates a mental state indicator, which is indicative of the likelihood of the subject suffering from one or more mental states at step 835.

At step 840, the server 310 stores the mental state indicator, typically as part of the subject data, optionally allowing the mental state indicator to be displayed, for example by forwarding this to the client device for display.

The process can end at this point, although more typically the server 310 compares the current mental state indicator to one or more previous mental state indicators for the subject at step 845, allowing a change in mental state to be assessed at step 850. This information can be displayed, for example as a longitudinal trend of changes in mental state indicator, optionally together with other accompanying information, such as details of interventions, allowing this information to be used by clinicians in tracking progression of mental states and the effectiveness of treatment.

Details of an example study will now be described.

In this example, Polysomnography (PSG) was conducted with similar procedures for multiple subjects.

To achieve this, qualified sleep technicians or research assistants placed electrodes according to the 10-20 system with at least the following derivations: 3 electroencephalogram channels (F3 or Fz, C3 or Cz and O1 or Oz), ground and reference channels, right and left electrooculograms, 2 chin electromyograms (EMG), 2 ECGs, nasal/oral thermistor and EMG leg electrode recordings. Sleep stages were visually scored by qualified sleep technicians at each sites, with participants being optionally asked to keep consistent sleep routines one week prior to polysomnography (confirmed by actigraphy and sleep diaries).

Raw ECG signal from each subject was processed to extract RR intervals. The results were visually inspected by trained operators to ensure quality r-wave extraction. The RR data was then temporally aligned with the sleep stage annotations. Quality filters were applied to remove patient data with insufficient RR quality stemming from poor electrode adhesion, electrical noise, motion artifacts, or other gaps in signal coverage.

The global sleep onset and wake periods derived from the EEG sleep annotations were used to define various sleep segments of interest for subsequent feature calculation. The following segments were leveraged to properly capture the heart-rate dynamics during the sleep cycle:

Immediate 20 minutes preceding sleep onset

Immediate 20 minutes following sleep onset

The overall sleep period from sleep onset to wake

The first half of the sleep period

The second half for the sleep period

The preceding 20 minutes prior to wake

For each segment, the RR and sleep data was processed to extract a bank of time & frequency related features.

Heart rate statistics:
Mean
Variance
Skew
Kurtosis
5th and 95th percentiles
Cumulative distribution function

Heart rate power spectral features including spectral power in classic bands:
Ultra low frequency defined as <0.003 Hz
Very low frequency as ≥0.003 Hz and <0.04 Hz
Low frequency defined as ≥0.04 Hz and <0.15 Hz
High-frequency defined as ≥0.15 Hz and <0.4 Hz
Ratio of low to high frequency

Heart rate variability features including:
Multi-scale entropy
SDANN—Standard deviation of the averages of NN intervals in all 5-minute segments
rMSSD—square root of the mean of the squares of differences between adjacent NN intervals

Sleep statistics
Total sleep duration ratio calculated as the sum of the sleep segments normalized by the window length
Number of sleep episodes
Mean sleep episode duration
Standard deviation of sleep episode durations

EEG-dependent metrics
Absolute time in each sleep stage (REM, Non-REM 1, 2, 3 & Wake)
Fractional time in each sleep stage (REM, Non-REM 1, 2, 3 & Wake)
Stage latency
Mean heart-rate in each sleep stage
Difference of mean heart-rates in each stage, e.g. mean HR in REM minus mean HR in NREM stage 1.

The metrics for each subject and associated label were used in a supervised machine learning framework leveraging a random forest classifier with 2000 trees. Initial investigations focused on evaluating and optimizing feature performance using simple model frameworks such as Naive Bayes classification and logistic regression. Additional performance was realized by enhancing model complexity through random forest and gradient boosted trees.

Initial model development leveraged the full complement of features, including ECG and EEG derived metrics, and the whole patient population. The classification model was trained on 70% of the overall data and tested on a hold-out 30% that was randomly selected. The Major depressive disorder (MDD) and control training groups were matched in terms of number of subjects. This model is subsequently referenced as the ‘ECG/EEG model.’

Subsequent modelling efforts focused on leveraging ECG-derived metrics with additional exclusion criteria applied. The combination of data source and exclusion criteria were chosen to better match the on-going prospective studies and target product configuration. This model is subsequently referenced as the ‘ECG model.’

To statistically characterize performance on the ‘ECG model,’ a 10 fold cross validation was repeated 10 times for a total of 100 generalization estimates. In each estimate, a 90/10 split of training to test data was utilized. Forests are generally robust to overfitting due to bagging, and cross validation ensures that no performance metric is derived from test data the model has trained on.

Performance of the EEG/ECG model on the hold-out set is shown in Table 1 below, whilst cross validated results for the ECG model, across the 100 generalization estimates over the 561 subjects are shown in Table 2.

TABLE 1

| Performance Metric | Mean |
|---|---|
| Accuracy | 86% |
| Sensitivity | 82% |
| Specificity | 88% |

TABLE 2

| Performance Metric | Mean | Std |
|---|---|---|
| AUROC | 79.5% | 5.6% |
| Accuracy | 73.5% | 5.5% |
| Sensitivity | 72.5% | 9.1% |
| Specificity | 74.1% | 7.0% |

Area under the receiver operating curve (AUROC) is an overall measure of classifier performance which represents the continuum of sensitivity and specificity tradeoffs. AUROC is most useful when directly comparing separate models outside of additional performance requirements like sensitivity or specificity. Accuracy is the fraction of samples correctly classified, which in a clinical setting is the probability that the model will output a correct result for a sample pulled from a similar demographic to the test set. Sensitivity (aka True Positive Rate) is the fraction of MDD samples correctly classified, which in a clinical setting is the probability of modeling someone with MDD as having MDD. Specificity (aka True Negative Rate) is the fraction of non-MDD samples correctly classified, which in a clinical setting is the probability of modeling someone who does not have MDD as not having MDD.

It is apparent that the EEG/ECG model serves an upper bound estimate of performance using physiologic data that can be captured in a controlled sleep lab environment. The sleep lab data acquisition modality is widely used but relatively expensive and inaccessible compared to widely available and low cost ambulatory ECG monitors. The developed ECG model represents classification performance of the target product configuration that can be deployed broadly at low-cost.

In any event, it will be apparent from this that the metrics proposed can be used to provide sufficient discriminatory performance in order to adequately classify subjects in respect of MDD.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers. As used herein and unless otherwise stated, the terms “approximately” or “about” mean±20%.

Persons skilled in the art will appreciate that numerous variations and modifications will become apparent. All such variations and modifications which become apparent to persons skilled in the art, should be considered to fall within the spirit and scope that the invention broadly appearing before described.

1. A monitoring system for generating a mental state indicator for use in identifying a mental state of a biological subject, the monitoring system including one or more electronic processing devices that:
a) obtain subject data indicative of at least a heart rate measured for the biological subject during at least part of a sleep episode;
b) analyze the subject data to determine at least one sleep segment selected from the group including:
i) n minutes preceding sleep onset;
ii) n minutes following sleep onset;
iii) the sleep episode;
iv) a first half of the sleep episode;
v) a second half of the sleep episode;
vi) n minutes prior to waking;
c) analyze the subject data to determine at least one metric for the at least one sleep segment, the at least one metric being selected from the metric group including:
i) a heart rate statistic metric selected from a heart rate statistic group including:
(1) a mean;
(2) a median;
(3) an average;
(4) a variance:
(5) a skew;
(6) a kurtosis;
(7) a percentile;
(8) a cumulative distribution function;
ii) a heart rate spectral power metric indicative of a spectral power in at least one frequency band selected from a frequency band group including:
(1) an ultra low frequency less than about 0.003 Hz;
(2) a very low frequency between about 0.003 Hz and about 0.04 Hz;
(3) a low frequency between about 0.04 Hz and about 0.15 Hz;
(4) a high-frequency between about 0.15 Hz and about 0.4 Hz;
iii) a heart rate variability metric selected from a heart rate variability group including:
(1) a multi-scale entropy;
(2) a standard deviation of average pulse intervals; and,
(3) square root of the mean of the squares of differences between adjacent pulse intervals; and,
d) apply the at least one metric to at least one computational model to determine an mental state indicator indicative of a mental state, the at least one computational model embodying a relationship between different mental states and one or more metrics, the at least one computational model being obtained by applying machine learning to reference metrics derived from heart rates measured for one or more reference subjects during at least part of a reference sleep period.

2. A monitoring system according to claim 1, wherein the one or more processing devices determine at least one of:
a) at least one metric for each of a plurality of sleep segments;
b) a plurality of metrics for at least one sleep segment; and,
c) a plurality of metrics for each of a plurality of sleep segments.

3. A monitoring system according to claim 1, wherein the subject data is indicative of a sleep state for the biological subject during at least part of each of a number of sleep episodes and wherein the at least one metric includes a sleep metric selected from a sleep metric group including:
a) a total sleep duration;
b) a number of sleep episodes;
c) a mean sleep episode duration; and,
d) a standard deviation of sleep episode durations.

4. A monitoring system according to claim 3, wherein the sleep state is derived from at least one of:
a) heart rate data indicative of the heart rate measured for the biological subject during at least part of a sleep period;
b) brain activity data indicative of brain activity measured for the biological subject during at least part of a sleep period; and,
c) activity data indicative of physical activity measured for the biological subject during at least part of a sleep period.

5. A monitoring system according to claim 1, wherein the subject data includes brain activity data indicative of brain activity measured for the biological subject during at least part of a sleep period and wherein the one or more processing devices:
a) analyze the brain activity data to determine at least one sleep stage selected from the sleep stage group including:
i) REM sleep;
ii) non-REM sleep stage 1;
iii) non-REM sleep stage 2;
iv) non-REM sleep stage 3; and,
v) awake; and,
b) determine at least one brain activity metric selected from a brain activity metric group including:
i) an absolute time in each sleep stage;
ii) a fractional time in each sleep stage;
iii) a sleep stage latency;
iv) a mean heart-rate in each sleep stage;
v) a difference of mean heart-rates in each sleep stage.

6. A monitoring system according to claim 1, wherein the one or more processing devices determine at least one of:
a) a plurality of metrics selected from:
i) a heart rate statistic metric group;
ii) a heart rate spectral power metric group;
iii) a heart rate variability metric group;
iv) a brain activity metric group; and,
v) a sleep metric group;
b) at least one of:
i) at least one of metric from each available group; and,
ii) at least two metrics from at least some available groups; and,
c) at least one of:
i) at least two metrics;
ii) at least three metrics;
iii) at least four metrics:
iv) at least five metrics;
v) at least six metrics;
vi) at least seven metrics;
vii) at least eight metrics;
viii) at least nine metrics; and,
ix) at least ten metrics.

7. (canceled)

8. (canceled)

9. A monitoring system according to claim 1, wherein the one or more processing devices:
a) determine one or more subject attributes from the subject data; and,
b) use the one or more subject attributes to at least one of:
i) apply the at least one computational model so that the at least one metric is assessed based on reference metrics derived for one or more reference subjects having similar attributes to the subject attributes;
ii) select a plurality of metrics; and,
iii) select at least one computational model.

10. (canceled)

11. (canceled)

12. A monitoring system according to claim 9, wherein the one or more subject attributes are selected from an attribute group including:
a) one or more subject characteristics selected from a characteristic group including:
i) a subject age;
ii) a subject height;
iii) a subject weight;
iv) a subject sex; and,
v) a subject ethnicity;
b) one or more possible mental states selected from a mental state group including:
i) healthy;
ii) abnormal;
iii) depression;
iv) anxiety;
v) panic disorder;
vi) obsessive compulsive disorder (OCD); and,
vii) schizophrenia;
c) one or more body states selected from a body state group including:
i) a healthy body state;
ii) an unhealthy body state; and,
iii) one or more disease states;
d) one or more medical symptoms selected from a medical symptom group including:
i) elevated temperature;
ii) coughing;
iii) sneezing;
iv) bloating;
v) abnormal bowel movement; and,
vi) nausea;
e) one or more perceived emotional states selected from an emotional state group including:
i) happy;
ii) sad;
iii) anxious;
iv) angry;
v) tired; and,
vi) shocked;
f) dietary information; and,
g) medication information.

13. A monitoring system according to claim 9, wherein the one or more processing devices determine the subject attributes at least one of:
a) by querying a subject medical history;
b) by receiving sensor data from a sensing device; and,
c) in accordance with user input commands.

14. A monitoring system according to claim 1, wherein the one or more processing devices:
a) compare at least one current metric determined for the subject during one or more current sleep episodes and at least one previous metric determined for the subject during one or more previous sleep episodes by:
i) applying the at least one current metric to the at least one computational model to determine a current mental state indicator indicative of a current mental state;
ii) applying the at least one previous metric to the at least one computational model to determine a previous mental state indicator indicative of a previous mental state; and,
iii) analysing a difference between the current and previous mental state indicators to determine the change in mental state; and,
b) using results of the comparison to track a change in mental state.

15. (canceled)

16. A monitoring system according to claim 14, wherein an intervention is performed between the previous and current sleep episodes and the one or more processing devices determine an indication of an effectiveness of the intervention based on the change in mental state.

17. A monitoring system according to claim 1, wherein the mental state indicator is at least one of:
a) indicative of at least one of:
i) a likelihood of the subject having a particular mental state; and,
ii) a likelihood of a severity of a particular mental state; and,
b) selected from the mental state group including:
i) normal;
ii) abnormal;
iii) depression;
iv) anxiety;
v) panic disorder;
vi) obsessive compulsive disorder (OCD); and,
vii) schizophrenia.

18. (canceled)

19. A monitoring system according to claim 1, wherein the system includes a monitoring device including:
a) at least one sensor; and,
b) a monitoring device processor that generates sensor data in accordance with signals from the at least one sensor, the sensor data being indicative of at least one of:
i) a heart rate of the subject;
ii) brain activity of the subject; and,
iii) physical activity of the subject.

20. A monitoring system according to claim 19, wherein at least one of:
a) the monitoring device is a wearable device; and,
b) the monitoring system includes a client device that:
i) receives sensor data from the monitoring device;
ii) generates captured subject data including:
(1) a subject identifier indicative of an identity of the subject; and,
(2) at least one of:
(a) heart rate data indicative of the measured heart rate;
(b) brain activity data indicative of measured brain activity; and,
(c) activity data indicative of measured physical activity; and,
iii) transfers captured subject data to the one or more processing devices, the one or more processing being responsive to the captured subject data to incorporate this into the subject data using the identifier.

21. (canceled)

22. (canceled)

23. A monitoring system according to claim 1, wherein the one or more processing devices at least one of:
a) display a representation of the mental state indicator;
b) store the mental state indicator for subsequent retrieval; and,
c) provide the mental state indicator to a client device for display.

24. A system according to claim 1, wherein the at least one computational model has a discriminatory performance at least one of:
a) based on at least one of:
i) an area under a receiver operating characteristic curve;
ii) an accuracy;
iii) a sensitivity; and,
iv) a specificity; and,
b) of at least 70%.

25. (canceled)

26. A method for generating an mental state indicator for use in identifying a mental state of a biological subject, the method including in one or more electronic processing devices:
a) obtaining subject data indicative of at least a heart rate measured for the biological subject during at least part of a sleep episode;
b) analyzing the subject data to determine at least one sleep segment selected from the group including:
i) i minutes preceding sleep onset;
ii) n minutes following sleep onset;
iii) the sleep period;
iv) a first half of the sleep period;
v) a second half of the sleep period;
vi) n minutes prior to waking;
c) analyzing the subject data to determine at least one metric for the at least one sleep segment, the at least one metric being selected from the metric group including:
i) a heart rate statistic metric selected from a heart rate statistic group including:
(1) a mean;
(2) a median;
(3) an average;
(4) a variance;
(5) a skew;
(6) a kurtosis;
(7) a percentile;
(8) a cumulative distribution function;
ii) a heart rate spectral power metric indicative of a spectral power in at least one frequency band selected from a frequency band group including:
(1) an ultra low frequency less than 0.003 Hz;
(2) a very low frequency between 0.003 Hz and 0.04 Hz;
(3) a low frequency between 0.04 Hz and 0.15 Hz;
(4) a high-frequency between about 0.15 Hz and about 0.4 Hz;
iii) a heart rate variability metric selected from a heart rate variability group including:
(1) a multi-scale entropy;
(2) a standard deviation of average pulse intervals; and,
(3) square root of the mean of the squares of differences between adjacent pulse intervals; and,
d) applying the at least one metric to at least one computational model to determine an mental state indicator indicative of a mental state, the at least one computational model embodying a relationship between different mental states and one or more metrics, the at least one computational model being obtained by applying machine learning to reference metrics derived from heart rates measured for one or more reference subjects during at least part of a reference sleep period.

27. A system for use in calculating at least one computational model, the at least one computational model being used for generating an mental state indicator for use in identifying a mental state of a biological subject, the system including one or more electronic processing devices that:
a) for each of a plurality of reference subjects:
i) obtain reference subject data indicative of:
(1) at least a heart rate measured the reference subject during at least part of a reference sleep episode; and,
(2) a diagnosed mental state of the reference subject;
ii) analyze the reference subject data to determine at least one sleep segment selected from the group including:
(1) n minutes preceding sleep onset;
(2) n minutes following sleep onset;
(3) the sleep episode;
(4) a first half of the sleep episode;
(5) a second half of the sleep episode;
(6) n minutes prior to waking;
iii) analyze the reference subject data to determine at least one reference metric for the at least one reference sleep segment, the at least one metric being selected from the metric group including:
(1) a heart rate statistic metric selected from a heart rate statistic group including:
(a) a mean;
(b) a median;
(c) an average;
(d) a variance;
(e) a skew;
(f) a kurtosis;
(g) a percentile;
(h) a cumulative distribution function;
(2) a heart rate spectral power metric indicative of a spectral power in at least one frequency band selected from a frequency band group including:
(a) an ultra low frequency less than about 0.003 Hz;
(b) a very low frequency between about 0.003 Hz and about 0.04 Hz;
(c) a low frequency between about 0.04 Hz and about 0.15 Hz;
(d) a high-frequency between about 0.15 Hz and about 0.4 Hz;
(3) a heart rate variability metric selected from a heart rate variability group including:
(a) a multi-scale entropy;
(b) a standard deviation of average pulse intervals; and,
(c) square root of the mean of the squares of differences between adjacent pulse intervals; and,
b) use the at least one reference metric and diagnosed mental state for a number of reference subjects to train at least one computational model, the at least one computational model embodying a relationship between different mental states and the at least one reference metric.

28. A system according to claim 27, wherein the one or more processing devices test the at least one computational model to determine a discriminatory performance of the model and wherein the discriminatory performance is at least one of:
a) based on at least one of:
i) an area under a receiver operating characteristic curve;
ii) an accuracy;
iii) a sensitivity; and,
iv) a specificity; and,
b) at least 70%.

29. (canceled)

30. (canceled)

31. (canceled)

32. A system according to claim 27, wherein the one or more processing devices:
a) select a plurality of reference metrics;
b) train at least one computational model using the plurality of reference metrics;
c) test the at least one computational model to determine a discriminatory performance of the model using reference subject data from a subset of the plurality of reference subjects; and,
d) if the discriminatory performance of the model falls below a threshold, at least one of:
i) selectively retrain the at least one computational model using a different plurality of reference metrics; and,
ii) train a different computational model.

33. A system according to claim 27, wherein the one or more processing devices:
a) select a plurality of combinations of reference metrics;
b) train a plurality of computational models using each of the combinations;
c) test each computational model to determine a discriminatory performance of the model; and,
d) selecting the at least one computational model with the highest discriminatory performance for use in determining an mental state indicator indicative of a mental state.

34. A system according to claim 27, wherein the one or more processing devices:
a) determine one or more reference subject attributes from the reference subject data;
b) train the at least one computational model using the one or more reference subject attributes by:
i) performing clustering using the using the reference subject attributes to determine clusters of reference subject having similar reference subject attributes; and,
ii) training the at least one computational model at least in part using the reference subject clusters.

35. (canceled)

36. A method for use in calculating at least one computational model, the at least one computational model being used for generating an mental state indicator for use in identifying a mental state of a biological subject, the method including, in one or more electronic processing devices:
a) for each of a plurality of reference subjects:
i) obtaining reference subject data indicative of:
(1) at least a heart rate measured the reference subject during at least part of a reference sleep episode; and,
(2) a diagnosed mental state of the reference subject;
ii) analyzing the reference subject data to determine at least one sleep segment selected from the group including:
(1) n minutes preceding sleep onset;
(2) n minutes following sleep onset;
(3) the sleep episode;
(4) a first half of the sleep episode;
(5) a second half of the sleep episode;
(6) n minutes prior to waking;
iii) analyzing the reference subject data to determine at least one reference metric for the at least one reference sleep segment, the at least one metric being selected from the metric group including:
(1) a heart rate statistic metric selected from a heart rate statistic group including:
(a) a mean;
(b) a median;
(c) an average;
(d) a variance;
(e) a skew;
(f) a kurtosis;
(g) a percentile;
(h) a cumulative distribution function;
(2) a heart rate spectral power metric indicative of a spectral power in at least one frequency band selected from a frequency band group including:
(a) an ultra low frequency less than about 0.003 Hz;
(b) a very low frequency between about 0.003 Hz and about 0.04 Hz;
(c) a low frequency between about 0.04 Hz and about 0.15 Hz;
(d) a high-frequency between about 0.15 Hz and about 0.4 Hz;
(3) a heart rate variability metric selected from a heart rate variability group including:
(a) a multi-scale entropy;
(b) a standard deviation of average pulse intervals; and,
(c) square root of the mean of the squares of differences between adjacent pulse intervals; and,
b) using the at least one reference metric and diagnosed mental state for a number of reference subjects to train at least one computational model, the at least one computational model embodying a relationship between different mental states and the at least one reference metric.
